(19) United States
(12) Patent Application Publication
(10) Pub. No.: US 2009/0214645 A1
(43) Pub. Date: Aug. 27, 2009

(54) PHARMACEUTICAL COMPOSITIONS COMPRISING A PH-DEPENDENT DRUG, A PH MODIFIER AND A RETARDING AGENT

(76) Inventors: Andrea Kramer, Reute (DE); Angelika Ries, Nenzlingen (CH); Stefanie Siepe, Freiburg (DE); Barbara Lückel, Lörrach (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/064,277

(22) PCT Filed: Aug. 22, 2006
(86) PCT No.: PCT/EP2006/008243

§ 371 Date: Oct. 1, 2008

(30) Foreign Application Priority Data
Aug. 22, 2005 (GB) 0517204.4
Sep. 8, 2005 (GB) 0518359.5

(51) Int. Cl. A61K 9/28; A61K 9/16

(52) U.S. Cl. 424/474; 424/490

(57) ABSTRACT

The present invention concerns pharmaceutical compositions comprising a pH-dependent drug compound and a pH modifier.

The present invention concerns pharmaceutical compositions comprising a pH-dependent drug compound, uses and processes for the manufacturing of such compositions.

pH-dependent drug compounds exhibit a significant pH-dependent solubility along the gastrointestinal tract. They are soluble at the low gastric pH expected in fasted healthy subjects. However, in a higher intestinal pH environment they may precipitate and/or dissolve incompletely. Additionally, food intake, medical treatment, and pathophysiological conditions may elevate the gastric pH and, consequently, drug dissolution may be decreased. Incomplete dissolution may result in highly variable inter-and intra patient bioavailability of the pH-dependent drug compound.

The incorporation of pH modifiers, e.g. organic acids, into an oral dosage form modulates the microenvironmental pH and thereby enhances the drug solubility and drug dissolution. In addition, pH-independent drug release can be achieved. However, pH modifiers typically exhibit a higher solubility at higher pH environments compared to the drug compound, diffuse out rapidly and consequently separate rapidly from the pH-dependent drug compound present in the solid dosage form. Polymers may be used to retard the rapid diffusion of pH modifiers thus maintaining the target pH inside the solid dosage form. However, the use of polymers typically result in modified release formulations, e.g. with a drug release over an extended period of time, e.g. over 10 hours and longer. Complete absorption of the drug compound from such modified release formulations may highly depend on the physiological conditions but also on the drug compound itself. For example, drug compounds that are absorbed only in a very specific, e.g. upper, part of the intestinal tract, e.g. the small intestine, may show a high inter- and intra subject variability and impaired bioavailability.

Accordingly, there is a need for pharmaceutical compositions comprising pH-dependent drug compounds with reduced inter- and intra subject variability and increased bioavailability. Surprisingly, the present inventors have identified improved pharmaceutical compositions comprising a pH-dependent drug compound and a pH modifier wherein the pH modifier is present inside the formulation e.g. over the entire dissolution time, e.g. simultaneously released together with the drug compound.

In one aspect, the present invention provides a pharmaceutical composition comprising a pH-dependent drug compound, a pH modifier and a retarding agent, e.g. a polymer, e.g. a water-soluble polymer, wherein the drug release from the pharmaceutical composition is completed after a maximum dissolution time of 4 hours, e.g., within a dissolution time of about 1 to 4 hours, e.g. within 1 to 2, or 1 to 3 hours, or about 2 to 4, e.g. 2 to 3 hours, e.g. upon contact with gastrointestinal juices.

In a further aspect of the invention, an additional enteric coating may be applied, e.g., to prevent any early diffusion of the drug and the acid in the stomach and/or to suppress individually varying stomach pH effects on dissolving the drug and the acid, e.g. to ensure uniform drug dissolution in the upper part of the intestinal tract, e.g. small intestine.

In yet a further aspect the present invention provides for an isolation coat between the acid core and the enteric coating, e.g., to provide for a dissolution of the enteric coat at an intestinal pH of about ≧5.5.

In yet a further aspect of the present invention, the pharmaceutical composition is in form of a multiparticulate system, e.g., minitablets or pellets. Such multiparticulate systems may show advantages over monolithic systems, e.g., improved transit reproducibility and/or high degree of dispersion in the digestive tract, resulting in reduced intra- and inter-subject variability and improved bioavailability.

These and other features, advantages and objects of the present invention will be further understood and appreciated by those skilled in the art by references to the following specification, claims and appended drawings.

### BRIEF DESCRIPTION OF THE DRAWINGS

The accompanying drawings, which are incorporated in and constitute a part of the specification, illustrate exemplary embodiments of the present invention.

FIG. 1 shows the simultaneous release rates of the drug and the pH modifier.

FIG. 2 shows the impact of pH modifiers on drug release.

FIG. 3 shows the drug release independent from the dissolution medium pH.

The compositions of the invention provide for short-duration modified release of a pH-dependent drug compound, in particular in the upper part of the intestinal tract, e.g. in the small intestine, showing reduced inter- and intra patient variability and improved bioavailability.

### DETAILED DESCRIPTION OF INVENTION

As used herein, the term “drug” means any compound, substance, drug, medicament or active ingredient having a therapeutic or pharmacological effect, and which is suitable for administration to a mammal, e.g., a human. Such drugs should be administered in a “therapeutically effective amount”.

As used herein, the term “therapeutically effective amount” refers to an amount or concentration which is effective in reducing, eliminating, treating, preventing or controlling the symptoms of a disease or condition affecting a mammal. The term “controlling” is intended to refer to all processes wherein there may be a slowing, interrupting, arresting or stopping of the progression of the diseases and conditions affecting the mammal. However, “controlling” does not necessarily indicate a total elimination of all disease and condition symptoms, and is intended to include prophylactic treatment.

The appropriate therapeutically effective amount is known to one of ordinary skill in the art as the amount varies with the therapeutic compound being used and the indication which is being addressed.

Drugs and salts thereof that are particularly suited for the present invention are those that are pH-dependent, in particular weakly basic drugs, e.g. any drug where the solubility difference between pH 1 and pH 6 is >100.

The drug may be present in an amount up to about 60% by weight of the composition, from about 1% to about 60% by weight of the composition. It is intended, however, that the choice of a particular level of drug will be made in accordance with factors well-known in the pharmaceutical arts, including mode of administration and the size and condition of the subject.

Suitable pH modifiers according to the invention include acids, e.g. inorganic acids, e.g. water-soluble inorganic acids that are solid at ambient temperature, for example sulfamic acid.

Suitable organic acids contain one or more acidic group, e.g. acidic groups selected from carboxylic and sulfonic acid groups, particularly those which are solid at ambient temperature.

Suitable water-soluble organic acids include water-soluble organic acids selected from mono, di- or polybasic carboxylic acids or mono, di or tri-sulfonic acids, e.g. which are solid at ambient temperature. Suitable solid water-soluble carboxylic acids include aliphatic mono or poly-carboxylic acids, e.g. containing from 2 to 8 carbon atoms, particularly from 2 to 6 carbon atoms, e.g. all- or tricarboxylic acids containing from 4 to 6, e.g. 4 carbon atoms, e.g. saturated or unsaturated. Examples of suitable solid water-soluble aliphatic mono-carboxylic acids include sorbic acid (2,4-hexandienoic acid). Examples of suitable solid water-soluble aliphatic di-carboxylic acids include adipic, malonic, succinic, glutaric, maleic or fumaric acid. The aliphatic carboxylic acid may be optionally substituted by one or more groups, e.g. 1, 2 or 3 groups, which may be the same or different, selected from carboxy, amino and hydroxy. Suitable substituted solid water-soluble aliphatic carboxylic acids include for example hydroxy substituted aliphatic mono-carboxylic acids such as gluconic acid, solid forms of lactic acid, glycolic acid or ascorbic acid; hydroxy substituted aliphatic di-carboxylic acids such as malic, tartaric, tartronic (hydroxymalonic), or mucic (galactaric) acid; hydroxy 2s substituted aliphatic tri-carboxylic acids, for example citric acid; or amino acids carrying an acidic side chain, such as glutamic acid or aspartic acid.

Suitable aromatic carboxylic acids include water-soluble aryl carboxylic acids containing up to 14 carbon atoms. Suitable aryl carboxylic acids comprise an aryl group, for example a phenyl or naphthyl group which carries one or more carboxyl groups, e.g. 1, 2 or 3 carboxy groups. The aryl group is optionally substituted by one or more groups, e.g. 1, 2 or 3 groups, which may be the same or different, selected from hydroxy, (1-4C) alkoxy, e.g. methoxy, and sulfonyl. Suitable aryl carboxylic acids include benzoic, phthalic, isophthalic, terephthalic or trimellitic acid (1,2,4-benzenetricarboxylic acid).

Preferably the pH modifier is selected from citric acid, fumaric acid, succininc acid, adipic acid and maleic acid. Preferably fumaric acid is used.

pH modifiers that are particularly suited for the present invention are those that change the microenvironmental pH towards being more acidic thereby increasing the release rate of the drug from the dosage form at pH values where the drug becomes difficultly soluble, e.g. insoluble.

The pH modifier comprises from about 1% to about 60% by weight of the composition, e.g., from about 10% to about 40% by weight of the composition. The ratio of pH modifier to drug compound in the compositions of the invention may be from about 0.2:1 to about 2:1, e.g. 1:1.

As retarding agents, polymers, e.g. water soluble polymers, e.g. cellulose derivatives, e.g., having a viscosity of greater than about 100 cps, e.g. having a viscosity of between about 100 and about 100,000 cps may be used. Preferably water-soluble polmers may be used.

Suitable polymers include but are not limited to cellulose derivatives, e.g. methyl cellulose, hydroxypropyl methyl cellulose, e.g. hydroxypropyl methyl cellulose k100LV, K 4 M, or hydroxypropyl methyl cellulose K 15 M, hydroxypropyl cellulose, hydroxyethyl cellulose, sodium-carboxy methyl cellulose, ethyl cellulose, e.g. ethyl cellulose 100, cellulose acetate, e.g. cellulose acetate CA-398-10 NF, cellulose acetate phthalate, cellulose acetate propionate, cellulose acetate butyrate, cellulose butyrate, cellulose nitrate, hydroxypropyl methyl cellulose phthalate, hydroxypropyl methyl cellulose acetate succinate, acryl derivatives, e.g. polyacrylates, e.g. cross-linked polyacrylates, methycrylic acid copolymers, vinyl polymers, e.g. polyvinyl pyrrolidones, polyvinyl acetates, or polyvinyl acetate phthalates and mixtures thereof, as marketed under the trade name Kollidon SR®, polyethylene glycols, polyanhydrides, polysaccharides, e.g. xanthans, e.g. xanthan gum, galactomannan, pectin, and alginates.

Preferred polymers include hydroxypropyl methyl cellulose, e.g. Methocel K100LV, Methocel K4M and Methocel K100M.

The polymer comprises from about 10% to about 60% by weight of the composition of the, e.g. from about 30% to about 60% by weight of the composition.

In certain exemplary embodiments of the present invention, the pharmaceutical composition may comprise additional excipients commonly found in pharmaceutical compositions, examples of such excipients include, but are not limited to glidants, lubricants, antioxidants, antimicrobial agents, enzyme inhibitors, stabilizers, preservatives, flavors, sweeteners and other components, e.g. as described in Handbook of Pharmaceutical Excipients, Rowe et al., Eds., 4^{th} Edition, Pharmaceutical Press (2003), which is hereby incorporated by reference.

These additional excipients may comprise from about 0.05-11% by weight of the total pharmaceutical composition, e.g. from about 0.5 to about 2% by weight of the total composition. Antioxidants, anti-microbial agents, enzyme inhibitors, stabilizers or preservatives typically provide up to about 0.05-1% by weight of the total pharmaceutical composition. Sweetening or flavoring agents typically provide up to about 2.5% or 5% by weight of the total pharmaceutical composition.

Suitable lubricant include, but are not limited to magnesium stearate, talc, hydrogenated castor oil, glycerylbehaptate, glycerolmonostearate, polyethylene glycol, ethylene oxide polymers, sodium lauryl sulfate, magnesium lauryl sulfate, sodium oleate, sodium stearyl fumarate, DL-leucine, colloidal silica, and others known in the art. The compositions of the invention may comprise between about 0 and 3%, e.g. between about 0.5 and 3%, e.g. 1% lubricant by weight of the composition.

Suitable fillers include, but are not limited to lactose, e.g. in an anhydrous or hydrated form, sugar, starches, e.g. corn, wheat, maize or potato starch, modified starches, e.g. starch hydrolysates or pregelatinized starch, mannitol, sorbitol, trehalose, maltose, glucose anhydrate; inorganic salts, e.g. calcium carbonate, magnesium carbonate, dibasic calcium phosphate, tribasic phosphate, or calcium sulfate, microcrystalline cellulose, cellulose derivates and others known in the art. The compositions of the invention may comprise between about 0 and 65%, e.g. between about 3 and 65% filler by weight of the composition.

Suitable glidants include but are not limited to Aerosil 200 or talc and others known in the art. The compositions of the invention may comprise between about 0 and 2% glidant by weight of the composition.

Suitable binders include but are not limited to polyvinylpyrrolidone (PVP), e.g. PVP K30 or PVP K12, as known and commercially available under the trade name Povidone® from the BASF company; or hydroxypropylmethylcellulose (HPMC), e.g. HMPC with a low apparent viscosity, e.g. below 100 cps as measured at 20° C. for a 2% by weight aqueous solution, e.g. below 50 cps, preferably below 20 cps, for example HPMC 3 cps, as known and commercially available under the name Pharmacoat® 603 from the Shin-Etsu company. The compositions of the invention may comprise between about 0 and 5%, e.g. between about 0.5 and 5% binder by weight of the composition.

Examples of antioxidants include, but are not limited to, ascorbic acid and its derivatives, tocopherol and its derivatives, butyl hydroxyl anisole and butyl hydroxyl toluene. Vitamin E as α-tocopherol is particularly useful.

In a further aspect of the present invention, there is provided a process for preparing a pharmaceutical composition, e.g., in the form of minitablets as described hereinabove, which process comprises mixing the active ingredient, the organic acid, the polymer, and any additional tableting excipients, and wet granulating with water or organic solvents. The dried granules for the preparation, e.g., in form of the minitablets, may be sieved through a 400 pm sieve. The outer phase, consisting of silicon dioxide, e.g. as available under the trade name Aerosil, and magnesium stearate, may be added and mixed thoroughly. The blend may be compressed into minitablets of a diameter of e.g. 1.5 to about 4 mm, e.g. 1.7 to 2 mm. Resulting minitablets may be encapsulated in a capsule, e.g. hard gelatin or starch capsule, or provided in a sachet.

In yet a further aspect of the present invention, there is provided a process for preparing a pharmaceutical composition, e.g. in the form of pellets as described hereinabove, which process comprises making a dry blend by mixing the active ingredient, the organic acid, the polymer and e.g. microcrystalline cellulose in a planetary mixer. Purified water may be added to give a wet mass that is subsequently extruded using a screen of a suitable size. The extrudates may be rounded in a spheroniser, thoroughly dried and sieved for suitable size selection, obtaining e.g. short duration modified release pellets.

In a further embodiment of the invention an enteric coating is applied to the minitablets or pellets.

As used herein the term “enteric coating” refers to a coating which protects the dosage form from dissolving already in the stomach, e.g. at pH 1 to 2 up to pH 5.

The enteric coating according to the invention may include the following (percentages relate to % of final coated minitablets or pellets)

2-40% polymers for enteric coating, e.g. Hydroxypropylmethylcellulose phthalate, e.g. as known under the trade name HP 50 or HP 55 and commercially available from Shin Etsu, Hydroxypropylmethylcellulose acetate succinate, e.g. as commercially available under the trade name Aqoat H, M, or L from Shin Etsu, Methyl acrylic acid—ethyl acrylic acid Copolymer (Methacrylic acid copolymer, USP), e.g. as commercially available under the trade name Eudragit L, S, L100-55, or L30D from Rbhm Pharma, Acryl-Eze from Colorcon, or Kollicoat MAE 30 DP from BASF, Cellulose acetatephthalate, e.g. as commercially available under the trade name Aquacoat CPD from FMC Biopolymer, or Polymer from Eastman Kodak, Polyvinylacetatephthalate, e.g. as commercially available under the trade name Sureteric from Colorcon

0-15% Polymers for subcoating (isolation coat between tablet core and enteric coat): Hydroxypropylmethylcellulose, e.g. as commercially available under the trade name Pharmacoat 603 or 606, ethylcellulose, e.g. as commercially available under the trade name Aquacoat ECD, FMC Biopolymer, or Surelease from Colorcon, and mixtures thereof with a ratio of Ethylcellulose:HPMC=1:1 up to 1:10, Polyvinylalcohol, e.g. as commercially available under the trade name Opadry II HP, type 85Ffrom Colorcon)

0-10% plasticizers, e.g. triacetine, triethylcitrate, PEG 4000, PEG 6000, PEG 8000, Diethylphthalate, Diethylsebacate, Acetyltriethylcitrate, etc.

0-15% antisticking agents, e.g. silicon dioxide, e.g. as commercially available under the trade name Aerosil 200, Syloid 244 FP, Talcum, Glycerolmonostearate, etc.

organic solvents or mixtures thereof with and without parts of water, e.g. ethanol, acetone, isopropanol, or water as needed to dissolve or disperse the coating polymers and excipients for coating solution

0-0.5% sodium hydroxide for redispersion of polymers for aqueous enteric coating suspensions, e.g. for redispersion of Eudragit L100-55.

In a further aspect the present invention provides for a process for coating of a pharmaceutical composition as defined herein, which process may comprise

In the case of organic enteric coating solution:

(1) dissolving the enteric coating polymer and the plasticizer in an organic solvent, and

(2) dispersing the antisticking agents.

In the case of coating from aqueous dispersions:

(1) dissolving or finely dispersing the plasticizer in water,

(2) dispersing the antisticking agent, and finally

(3) adding the reconstituted suspension (e.g. Aqoat or Eudragit L 100-55,) or the commercially available aqueous polymer dispersion (e.g. Eudragit L 30D, Acryl-Aze, Kollicoat MAE 30 D).

Optionally an isolation coat may be applied comprising, e.g., an aqueous solution of a suitable polymer, e.g. Hydroxypropylmethylcellulose (HPMC) (4-8%), plasticizer (0-3%) and antisticking agent (0-3%). An aqueous ethylcellulose dispersion, e.g. Aquacoat ECD or Surelease, may be added in the range of 1:10 up to 1:1 (Ethylcellulose : HPMC) to improve the isolation effect of the subcoating. Based on the minitablet/pellet size the total amount of subcoat applied may be between 3-15% (more probably 5-10%). Polyvinylalcohol (Opadry I1 HP) in a range of 2-10% of core weight may be employed for an effective subcoating. In a further aspect, a HPMC subcoat may be applied in form of an organic suspension in ethanol/acetone 1:1 (about 6-10% polymer per solvent) without any further additives.

The enteric coating and/or subcoat may be applied using a pan coater or fluidized bed coater with or without Wurster principle up to a coating layer between 2 and 45% by weight of the dosage form, e.g. between about 10-25%, e.g. for large tablets, e.g. having a diameter of between about 5 and 15 mm, and between about 20-40%, e.g. for small tablets, e.g. minitablets, e.g. having a diameter of between about 1.5 and 4 mm, e.g between 1.7 and 2 mm. The subcoating layer may comprise between about 2-15% by weight of the dosage form, e.g. between about 4-10%, e.g. for large tablets, and between about 8-15%, e.g. for small tablets, e.g. minitablets, or pellets. The enteric coating layer may comprise between about 540% by weight of the dosage form, e.g. between about 8-20%, e.g. for large tablets, and between about 15-30%, e.g. for small tablets, e.g. minitablets, or pellets: The layer may depend on the minitablet/pellet size to assure an enteric resistance for 1-3 hours in artificial gastric juice or 0.1 n HCL solution (acc. to Ph Eur. or USP). Additionally, swelling of the core during gastric resistance test should be reduced to a minimum.

In a further aspect the present invention provides for a pharmaceutical composition as defined hereinabove providing a complete drug release, e.g. complete disintegration of the dosage form, within up to 4 hours, e.g. upon contact with gastrointestinal juices, e.g in the stomach in the case of non-enteric coated dosage forms, or in the upper part of the intestinal tract, e.g. the small intestine, in the case of enteric coated dosage forms. For example complete drug release may be provided within about 1 to 4 hours, e.g. within 1 to 3 or 1 to 2 hours, or within about 2 to 4, e.g. 2 to 3 hours.

The utility of all the pharmaceutical compositions of the present invention may be observed in standard clinical tests in, e.g., known indications of drug dosages giving therapeutically effective blood levels of drug, e.g., using dosages in the range of 2.5-1000 mg of drug per day for a 75 kg mammal, e.g., adult and in standard animal models. The increased bioavailability of the drug provided by the compositions may be observed in standard animal tests and in clinical trials.

The following examples are illustrative, but do not serve to limit the scope of the invention described herein. The examples are meant only to suggest a method of practicing the present invention. Quantities of ingredients, represented by percentage by weight of the pharmaceutical composition, used in each example are set forth in the respective tables located after the respective descriptions.

### EXAMPLES

### 1. Minitablets

The drug, the organic acid, the polymer, and any additional tableting excipients, are mixed and wet granulated with water or organic solvents in a mortar. After drying at 40° C., the dried granules for the preparation of the minitablets are sieved through a 400 μm, sieve. The outer phase, comprising silicon dioxide and magnesium stearate, is added and mixed thoroughly. The blend is compressed into minitablets having a diameter of 1.7 to 2 mm.

| | [%] | mg/capsule |
|---|---|---|
| Example 1 | | |
| Methocel K100LV | 30.00 | 75.00 |
| Dipyridamole | 10.00 | 25.00 |
| Fumaric acid | 20.00 | 50.00 |
| Lactose 200 mesh | 34.00 | 85.66 |
| HPMC 3 cps | 2.67 | 6.68 |
| Mg-Stearate | 1.33 | 3.33 |
| Aerosil 200 | 2.00 | 5.00 |
| | 100.00 | 250.00 |
| Example 2 | | |
| Methocel K100LV | 30.00 | 75.00 |
| Dipyridamole | 10.00 | 25.00 |
| Fumaric acid | 40.00 | 100.00 |
| Lactose 200 mesh | 14.00 | 35.00 |
| HPMC 3 cps | 2.67 | 6.68 |
| Mg-Stearate | 1.33 | 3.33 |
| Aerosil 200 | 2.00 | 5.00 |
| | 100.00 | 250.00 |
| Example 3 | | |
| Methocel K100LV | 30.00 | 75.00 |
| Lactose 200 mesh | 54.00 | 135.00 |
| Dipyridamole | 10.00 | 25.66 |
| Fumaric acid | 0.00 | 0.00 |
| HPMC 3 cps | 2.67 | 6.68 |
| Mg-Stearate | 1.33 | 3.33 |
| Aerosil 200 | 2.00 | 5.00 |
| | 100.00 | 250.00 |
| Example 4 | | |
| Methocel K100LV | 30.00 | 75.00 |
| Dipyridamole | 10.00 | 25.00 |
| Succinic acid | 20.00 | 50.00 |
| Lactose 200 mesh | 34.00 | 85.66 |
| HPMC 3 cps | 2.67 | 6.68 |
| Mg-Stearate | 1.33 | 3.33 |
| Aerosil 200 | 2.00 | 5.00 |
| | 100.00 | 250.00 |
| Example 5 | | |
| Methocel K100LV | 30.00 | 75.00 |
| Dipyridamole | 10.00 | 25.00 |
| Succinic acid | 40.00 | 100.00 |
| Lactose 200 mesh | 14.00 | 35.00 |
| HPMC 3 cps | 2.67 | 6.68 |
| Mg-Stearate | 1.33 | 3.33 |
| Aerosil 200 | 2.00 | 5.00 |
| | 100.00 | 250.00 |
| Example 6 | | |
| Methocel K4M | 30.00 | 75.00 |
| Dipyridamole | 10.00 | 25.00 |
| Fumaric acid | 20.00 | 50.00 |
| Lactose 200 mesh | 34.00 | 84.99 |
| HPMC 3 cps | 2.67 | 6.68 |
| Mg-Stearate | 1.33 | 3.33 |
| Aerosil 200 | 2.00 | 5.00 |
| | 100.00 | 250.00 |

### 2. Pellets:

A dry blend is made by mixing the active ingredient, the organic acid, the polymer and e.g. microcrystalline cellulose in a planetary mixer. Purified water is added to give a wet mass that is subsequently extruded using a screen of a suitable size. The extrudates are rounded in a spheroniser, thoroughly dried and sieved for suitable size selection, obtaining short duration modified release pellets.

| Example 7 | % w/w | mg/capsule |
|---|---|---|
| Methocel K100M | 30 | 75 |
| Dipyridamole | 10 | 25 |
| Fumaric acid | 20 | 50 |
| Lactose | 40 | 100 |
| monohydrate 200 | | |
| mesh | | |

### 3. Enteric Coating:

### Examples 1 to 4

### Preparation of Subcoat:

The subcoat is prepared from an aqueous solution of polymer, plasticizer and antisticking agent. Optionally an aqueous ethylcellulose dispersion (Aquacoat ECD or Surelease) is added. Alternatively, an organic suspension of polymer in water or ethanol/acetone 1:1 is prepared.

### Preparation of Enteric Coat:

In case of an organic enteric coating solution, after dissolving the enteric coating polymer and the plastisizer in organic solvents, the antisticking agents are dispersed. In case of a coating from aqueous dispersions, the plasticizer is dissolved or finely dispersed in water, the antisticking agent is dispersed, and finally the reconstituted suspension (i.e. Aqoat or Eudragit L 100-55,) or the commercially available aqueous polymer dispersion (Eudragit L 30D, Acryl-Aze, Kollicoat MAE 30 D) are added.

### Coating Process:

The coating is applied using a pan coater or fluidized bed coater with or without Wurster principle up to a coating layer between 2 and 45% (about 10-25% for large tablets and 20-40% for small tablets/minitablets). Subcoating layer: 2-15% (large tablets 4-10%, minitablets/pellets: 8-15%)/enteric coating layer: 5-40%( large tablets: 8-20%, minitablets/pellets: 15-30%) The layer depends on the minitablet/pellet size to assure an enteric resistance for 1-3 hours in artificial gastric juice or 0.1 n HCL solution (acc. to Ph Eur. or USP). Additionally, swelling of the core during gastric resistance test is reduced to a minimum.

| | | | mg/ | mg/ |
|---|---|---|---|---|
| | parts | % | 250 mg core | 8 mg core |
| Example 1 | | | | |
| Subcoat | | | | |
| HPMC 3 cps | 5.0 | 25.0 | 12.50 | 0.80 |
| Trietylcitrate | 0.5 | 2.5 | 1.25 | 0.08 |
| Talc | 0.5 | 2.5 | 1.25 | 0.08 |
| Water q.s. | | | | |
| Enteric coat | | | | |
| Eudragit L 30 D (dry) | 10.0 | 50.0 | 25.00 | 1.60 |
| PEG 6000 | 2.0 | 10.0 | 5.00 | 0.32 |
| Sytoid 244 FP | 2.0 | 10.0 | 5.00 | 0.32 |
| Water q.s | | | | |
| Total (dry) | 20.0 | 100.0 | 50.00 | 3.20 |
| Example 2 | | | | |
| Subcoat | | | | |
| HPMC 3 cps | 6.0 | 26.67 | 15.0 | 0.960 |
| Aquacoat ECD (dry) | 2.0 | 8.89 | 5.0 | 0.320 |
| Trietylcitrate | 0.6 | 2.67 | 1.5 | 0.096 |
| GLycerinemonostearate | 0.4 | 1.77 | 1.0 | 0.064 |
| Water q.s. | | | | |
| Enteric coat | | | | |
| HPMC AS (Aqoat) MF | 10.0 | 44.44 | 25.00 | 1.600 |
| Triethylcitrate | 2.5 | 11.11 | 6.25 | 0.400 |
| Talc | 1.0 | 4.44 | 2.5 | 0.160 |
| Water q.s. | | | | |
| Total (dry) | 22.5 | 100.0 | 56.25 | 3.600 |
| Example 3 | | | | |
| Subcoat | | | | |
| HPMC 3 cps | 5.0 | 32.5 | 12.5 | 0.80 |
| Ethanol/Acetone 1:1: | | | | |
| q.s. | | | | |
| Enteric coat | | | | |
| HP 50 | 8.0 | 51.9 | 20.0 | 1.28 |
| Triacetine | 0.8 | 5.2 | 2.0 | 0.13 |
| Aerosil 200 | 1.6 | 10.4 | 4.0 | 0.26 |
| Ethanol/Acetone 1:1: | | | | |
| q.s. | | | | |
| Total (dry) | 15.4 | 100.0 | 38.5 | 2.47 |
| Example 3b | | | | |
| Enteric coat | | | | |
| HP 50 | 10 | 71.43 | 25.0 | 1.60 |
| Dieethylsebacate | 1 | 7.14 | 2.5 | 0.16 |
| Talc | 3 | 21.43 | 7.5 | 0.48 |
| Ethanol/Acetone 1:1 | | | | |
| q.s. | | | | |
| Total dry | 14 | 100.0 | 34.0 | 2.24 |
| Example 4 | | | | |
| Subcoat | | | | |
| Opadry II HP | 4.00 | 21.46 | 10.00 | 0.640 |
| Water q.s. | | | | |
| Enteric coat | | | | |
| Eudragit L100-55 | 10.00 | 53.65 | 25.00 | 1.600 |
| Sodium hydroxide | 0.14 | 0.75 | 0.35 | 0.022 |
| Triethylcitrate | 2.50 | 13.41 | 6.25 | 0.400 |
| Syloid 244 FP | 2.00 | 10.73 | 5.00 | 0.320 |
| Water q.s | | | | |
| Total dry | 18.64 | 100.0 | 46.60 | 2.982 |
| Example 4b | | | | |
| Enteric coat | | | | |
| Eudragit L 100-55 | 10 | 76.92 | 25.0 | 1.60 |
| Triethylcitrate | 1 | 7.69 | 2.5 | 0.16 |
| Syloid 244 FP | 2 | 15.38 | 5.0 | 0.32 |
| Isopropanol/Water | | | | |
| 97:3 q.s | | | | |
| Total dry | 13 | 100.0 | 32.5 | 2.08 |

### 4. In Vitro Dissolution Studies

Dissolution studies are conducted in USP 1 apparatus (100 rpm, 37° C., and 500 ml dissolution medium). Tablets are exposed to a constant pH medium (phosphate buffer, pH=6.8) for a time interval of 6 hours. 0.1% SDS is added to the buffer to create sink conditions. At predetermined intervals samples are withdrawn from the dissolution medium and filtered through 0.45 μm membrane filters. Dipyridamole is analyzed spectrophotometrically at a wavelength of 410 nm (Perkin Elmer UV/VIS), whereas acid release is quantified by HPLC. An equivalent amount of fresh buffer is added to maintain a constant dissolution volume. All experiments are performed in triplicate.

### HPLC-Assay

The chromatography is carried out on Agilent HPLC, HP1100, equipped with Chromeleon software for data analysis. During the first 8 minutes, the mobile phase consists of 0.1 M NH₄H₂PO₄ buffer adjusted to pH 2.7 with phosphoric acid. Subsequently, a gradient (acetonitrile/NH₄H₂PO₄ buffer (pH 2.7)) is used to remove possibly remaining drug compound completely. Separation is achieved by using an lnertsil C8-3.5 μm, 4.6*150 mm (Erchatech AG, Switzerland). A flow rate of 1 ml/min, an injection volume of 5 μL (FA) and 10 μL (CA and SA), and run times of 15 min are applied. Chromatograms are recorded at 210 nm.

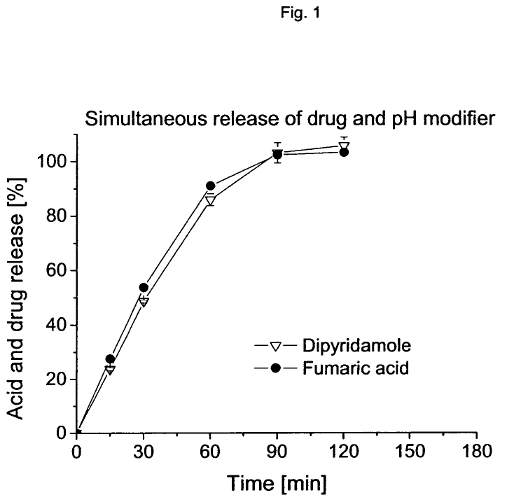

4.1 Simultaneous release of drug compound Dipyridamole 10% (Dipyridamole shown as open triangles in FIG. 1) and fumaric acid (variable concentration) (fumaric acid shown as filled circles in FIG. 1) from Methocel K4M 30% based tablets is demonstrated in FIG. 1.

Dissolution conditions: a) Phosphate buffer pH 6.8, SDS 0.1%; b) 0.01 N HCl. The drug is analysed by UV spectrometry (wavelength: 410 nm), fumaric acid by HPLC

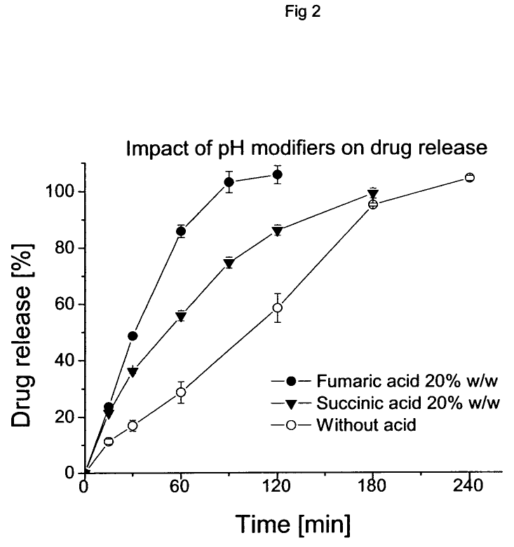

4.2 The impact of pH modifiers (fumaric acid 20% w/w shown as filled circles, succinic acid 20% w/w shown as filled triangle, and without acid shown as open circles in FIG. 2) on release of drug compound (Dipyridamole 10%) from Methocel K100LV 30% based tablets is demonstrated in FIG. 2. Dissolution conditions: Phosphate buffer pH 6.8; SDS 0.1%. The drug is analysed by UV spectrometry (wavelength: 410 nm).

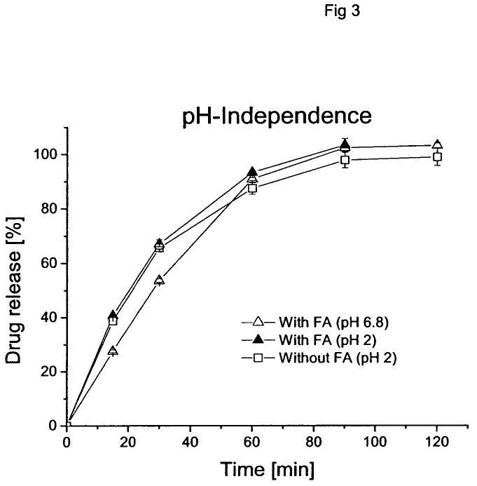

4.3 pH-independence of drug release with and without fumaric acid (with fumaric acid (pH 6.8) shown as open triangle, with fumaric acid (pH 2) shown as filled triangle and without fumaric acid (pH 2) shown as open squares in FIG. 3) is demonstrated in FIG. 3. Dissolution conditions: a) Phosphate buffer pH 6.8, SDS 0.1%; b) 0.01 N HCl

The drug is analysed by UV spectrometry (wavelength: 410 nm).

The dissolution of a pH dependent drug compound is enhanced at higher pH environments. Incorporation of pH modifiers, e.g. fumaric acid, may shift the pH inside and in the closest vicinity of the solid dosage form, e.g. minitablets/pellets, towards being more acidic, thus enhancing the drug solubility and dissolution. The polymer helps to maintain the target pH within and around the dosage form. Simultaneous release rates of the drug and the pH modifier is achieved throughout the entire dissolution.

It is understood that while the present invention has been described in conjunction with the detailed description thereof that the foregoing description is intended to illustrate and not limit the scope of the invention, which is defined by the scope of the following claims. Other aspects, advantages and modifications are within the scope of the claims.

1. A pharmaceutical composition comprising a pH-dependent drug compound, a pH modifier and a retarding agent, wherein the drug release from the pharmaceutical composition is completed after a maximum dissolution time of between 1 and 4 hours.

2. The pharmaceutical composition of claim 1 wherein the drug release from the pharmaceutical composition is completed after a maximum dissolution time of between 2 and 4 hours.

3. The pharmaceutical composition of claim 1 wherein the pH modifier is an organic acid selected from the group consisting of citric acid, fumaric acid, succininc acid, adipic acid and maleic acid.

4. The pharmaceutical composition of claim 1 wherein the retarding agent is a polymer, for example a water-soluble polymer.

5. The pharmaceutical composition of claim 1 wherein drug and pH modifier are simultaneously released from the pharmaceutical composition.

6. The pharmaceutical composition of claim 1 comprising an enteric coating.

7. The pharmaceutical composition of claim 6 further comprising an isolation coat between the acid core and the enteric coating.

8. The pharmaceutical composition of claim 6 in a form selected from the group consisting of minitablets and pellets.

9. A pharmaceutical composition comprising a pH dependent drug, fumaric acid and a cellulose derivative.

10. (canceled)

11. (canceled)

12. (canceled)

13. A method of reducing intra- and intersubject variability and improved bioavailability comprising the step of administering a pharmaceutical composition having a pH-dependent drug compound, a pH modifier and a retarding agent, wherein the drug release from the pharmaceutical composition is completed after a maximum dissolution time of between 1 and 4 hours.

14. The method of providing complete drug release from the pharmaceutical composition in the upper part of the intestinal tract, comprising the step of administering a pharmaceutical composition having a pH-dependent drug compound, a pH modifier and a retarding agent, wherein the drug release from the pharmaceutical composition is completed after a maximum dissolution time of between 1 and 4 hours.
